(19) United States
(12) Patent Application Publication
(10) Pub. No.: US 2021/0138230 A1
(43) Pub. Date: May 13, 2021

(54) TERMINAL DEVICE, ELECTRICAL TREATMENT DEVICE, AND TREATMENT SYSTEM

(71) Applicant: OMRON HEALTHCARE Co., Ltd., Kyoto (JP)

(72) Inventors: Hideaki YOSHIDA, Kyoto (JP); Mitsuru SAMEJIMA, Kyoto (JP); Tsuyoshi OGIHARA, Kyoto (JP); Tamaki ITO, Kyoto (JP); Sayuri NATSUKAWA, Kyoto (JP); Akira SOUMI, Kyoto (JP); Masaaki NONOYAMA, Kyoto (JP)

(21) Appl. No.: 17/154,590

(22) Filed: Jan. 21, 2021

Related U.S. Application Data
(63) Continuation of application No. PCT/JP2019/027766, filed on Jul. 12, 2019.

(30) Foreign Application Priority Data
Jul. 30, 2018 (JP) 2018-142547

(51) Int. Cl. A61N 1/04; A61N 1/36; G06F 3/041

(52) U.S. Cl. CPC A61N 1/0408; G06F 2203/04105; G06F 3/0412; A61N 1/36034

(57) ABSTRACT

A terminal device (10) including a touch panel (155) is configured to be able to communicate with an electrical treatment device (20) including a plurality of electrodes to be brought into contact with a site of the body of a user. The terminal device (10) includes a display control unit (407) configured to display a predetermined image on a display screen of the touch panel (155), a selection unit (403) configured to select a set of electrodes constituted of two electrodes among the plurality of electrodes in response to a touch operation performed on the predetermined image displayed on the display screen, and an instruction unit (405) configured to instruct the electrical treatment device (20) to apply a pulse voltage to the selected set of electrodes.

### CROSS-REFERENCE TO RELATED APPLICATIONS

This application is the U.S. national stage application filed pursuant to 35 U.S.C. 365(c) and 120 as a continuation of International Patent Application No. PCT/JP2019/027766, filed Jul. 12, 2019, which application claims priority from Japanese Patent Application No. 2018-142547, filed Jul. 30, 2018, which applications are incorporated herein by reference in their entireties.

### TECHNICAL FIELD

The present disclosure relates to a terminal device, an electrical treatment device, and a treatment system.

### BACKGROUND ART

A known electrical treatment device provides electrical stimulation in such a manner that a plurality of electrodes are set to be in contact with a surface of the body such as the abdomen and the back, and then electrical signals are output to muscles in the body via the electrodes.

For example, JP 10-118199 A (Patent Document 1) discloses a low-frequency treatment device in which at least one stimulation pattern is sequentially generated by a controller program. This low-frequency treatment device includes three or more electrodes, and the electrodes are disposed symmetrically on a base sheet body.

### CITATION LIST

### Patent Literature

Patent Document 1: JP 10-118199 A

### SUMMARY OF INVENTION

### Technical Problem

The low-frequency treatment device according to Patent Document 1 provides various stimulation patterns by changing a different electrode and an indifferent electrode every constant time period among five electrodes. For such a low-frequency treatment device having a plurality of electrodes, there is a need of a user to generate a desired stimulation pattern by selecting the electrodes from among the plurality of electrodes by intuitive operation. However, in Patent Document 1, user-selectable stimulation patterns are limited to the stimulation patterns having been stored in advance, and any technique satisfying the above need is not disclosed or suggested.

An object in an aspect of the present disclosure is to provide a terminal device, an electrical treatment device, and a treatment system configured to enable treatment in a desired stimulation pattern by selecting electrodes by a touch operation.

### Solution to Problem

In an example of the present disclosure, a terminal device including a touch panel is configured to be able to communicate with an electrical treatment device including a plurality of electrodes to be brought into contact with a site of the body of a user. The terminal device includes a display control unit configured to display a predetermined image on a display screen of the touch panel, a selection unit configured to select a set of electrodes constituted of two electrodes among the plurality of electrodes in response to a touch operation performed on the predetermined image displayed on the display screen, and an instruction unit configured to instruct the electrical treatment device to apply a pulse voltage to the selected set of electrodes.

According to the above configuration, the selection of the electrodes by the touch operation makes it possible to perform the treatment with a desired stimulation pattern.

In another example of the present disclosure, the predetermined image includes a plurality of electrode images corresponding to the plurality of electrodes respectively. The selection unit extracts a set of electrode images constituted of two electrode images among the plurality of electrode images in response to a touch operation performed on the plurality of electrode images, and selects the set of electrodes corresponding to the set of electrode images.

According to the above-described configuration, the plurality of electrode images respectively corresponding to the plurality of electrodes are displayed, so that the desired electrodes may be easily selected.

In another example of the present disclosure, the selection unit extracts, from among the electrode images displayed on the display screen, two electrode images closest to a touch position specified by the touch operation as the set of electrode images.

According to the above configuration, a user may receive electrical stimulation between the electrodes corresponding to the set of electrode images without having to touch the two electrode images individually.

In another example of the present disclosure, the selection unit extracts, from among the electrode images, a first electrode image closest to a first touch position specified by the touch operation and a second electrode image closest to a second touch position specified by the touch operation, as the set of electrode images.

According to the configuration discussed above, the user may freely select any two electrode images from among the electrode images. Thus, for example, by touching two electrode images that are not adjacent to each other, electrical stimulation between the electrodes corresponding to the two electrode images may be received.

In another example of the present disclosure, the plurality of electrodes are formed to have a honeycomb shape. Each of the electrode images is arranged in a honeycomb-like pattern on the display screen of the touch panel.

According to the above-described configuration, the plurality of electrodes included in the electrical treatment device may be efficiently arranged. In addition, since each of the electrode images is arranged in the honeycomb-like pattern, each electrode image is in contact with multiple electrode images (six at the most), whereby an improvement in operability at the time of continuously touching the electrode images by the touch operation is also obtained.

In another example of the present disclosure, a plurality of regions in the predetermined image are associated with the plurality of electrodes, respectively. The selection unit extracts a set of regions constituted of two regions among the plurality of regions in response to a touch operation performed on the predetermined image, and selects the set of electrodes corresponding to the set of regions.

According to the above-described configuration, by touching the region in the predetermined image displayed on the display screen of the touch panel, the electrode corresponding to the touched region can be easily selected.

In another example of the present disclosure, the selection unit extracts, from among the plurality of regions, two regions closest to a touch position specified by the touch operation as the set of regions.

According to the above configuration, a user may receive electrical stimulation between the electrodes corresponding to the set of regions without having to touch the two regions individually.

In another example of the present disclosure, the selection unit extracts, from among the plurality of regions, a first region closest to a first touch position specified by the touch operation and a second region closest to a second touch position specified by the touch operation, as the set of regions.

According to the above-discussed configuration, by touching two regions separated from each other, the user may receive electrical stimulation between the electrodes corresponding to the two regions.

In another example of the present disclosure, the terminal device further includes a pressing force detection unit configured to detect a pressing force applied to the touch panel by the touch operation, and an adjustment unit configured to adjust an electrical stimulation intensity provided to the site based on the pressing force when the set of electrodes is selected. The instruction unit instructs the electrical treatment device to apply a pulse voltage corresponding to the adjusted electrical stimulation intensity to the set of electrodes.

According to the above-described configuration, by adjusting the pressing force when performing the touch operation, the electrical stimulation intensity provided to a treatment site of the user may be easily changed.

In another example of the present disclosure, the adjustment unit increases the electrical stimulation intensity as the pressing force increases.

According to the above-described configuration, the user can increase the electrical stimulation intensity in proportion to the strength of the pressing force against the touch panel, which makes it possible to provide an intuitive and easy-to-follow operation scheme.

In another example of the present disclosure, the terminal device further includes an information storage unit configured to store pattern information including each of a plurality of the sets of electrodes selected by the selection unit, and an order in which the sets of electrodes are selected, in accordance with a series of touch operations. The instruction unit instructs the electrical treatment device to apply a pulse voltage to each set of electrodes based on the pattern information stored in the information storage unit.

According to the above-described configuration, a desired stimulation pattern corresponding to a series of touch operations is stored in the information storage unit. Thus, the user can receive treatment based on the stimulation pattern without performing again the same series of touch operations.

In another example of the present disclosure, an electrical treatment device having a touch panel includes: a plurality of electrodes to be brought into contact with a site of the body of a user; a display control unit configured to display a predetermined image on a display screen of the touch panel; a selection unit configured to select a set of electrodes constituted of two electrodes among the plurality of electrodes in response to a touch operation performed on the predetermined image displayed on the display screen; and a treatment execution unit configured to perform treatment on the site by applying a pulse voltage to the selected set of electrodes.

According to the above configuration, the selection of the electrodes by the touch operation makes it possible to perform the treatment with a desired stimulation pattern.

In another example of the present disclosure, a treatment system includes: an electrical treatment device including a plurality of electrodes to be brought into contact with a site of the body of a user; and a terminal device including a touch panel and configured to be able to communicate with the electrical treatment device. The terminal device further includes a display control unit configured to display a predetermined image on a display screen of the touch panel, a selection unit configured to select a set of electrodes constituted of two electrodes among the plurality of electrodes in response to a touch operation performed on the predetermined image displayed on the display screen, and an instruction unit configured to instruct the electrical treatment device to apply a pulse voltage to the selected set of electrodes.

According to the above configuration, the selection of the electrodes by the touch operation makes it possible to perform the treatment with a desired stimulation pattern.

### Advantageous Effects of Invention

According to the present disclosure, the selection of the electrodes by the touch operation makes it possible to perform the treatment with a desired stimulation pattern.

### BRIEF DESCRIPTION OF DRAWINGS

FIG. 1 is a diagram illustrating a treatment system according to the present embodiment.

FIG. 2 is a block diagram illustrating an example of a hardware configuration of a terminal device according to a first embodiment.

FIG. 3 is a perspective view illustrating an example of a configuration of an electrical treatment device according to the first embodiment.

FIG. 4 is a perspective view illustrating a state in which a main body portion provided in an electrical treatment device according to the first embodiment is separated from a holder and a pad 2.

FIG. 5 is a diagram illustrating an example of an electrode structure formed in a pad according to the first embodiment.

FIG. 6 is a diagram for describing parameters of a pulse voltage waveform.

FIG. 7 is a diagram illustrating an example of an operation screen for selecting an electrode to be applied with a pulse voltage.

FIG. 8 is a diagram illustrating another example of an operation screen for selecting an electrode to be applied with a pulse voltage.

FIG. 9 is a diagram illustrating an example of an operation screen in a case in which a series of touch operations is performed.

FIG. 10 is a block diagram illustrating an example of a functional configuration of a terminal device according to the first embodiment.

FIG. 11 is a flowchart illustrating an example of a processing procedure of a terminal device according to the first embodiment.

FIG. 12 is a diagram illustrating an example of an appearance of an electrical treatment device according to a second embodiment.

FIG. 13 is a block diagram illustrating an example of a hardware configuration of an electrical treatment device according to the second embodiment.

FIG. 14 is a diagram illustrating an example of an operation screen for selecting an electrode to be applied with a pulse voltage in another embodiment.

### DESCRIPTION OF EMBODIMENTS

Embodiments of the present invention will be described below with reference to the drawings. In the following description, like components are given like numerals. Names and functions thereof are also the same. Thus, the detailed description of such components is not repeated.

### Application Example

An application example of the present invention will be described with reference to FIG. 1. FIG. 1 is a diagram illustrating a treatment system 1000 according to the present embodiment. Referring to FIG. 1, the treatment system 1000 includes a terminal device 10, which is a user terminal, an electrical treatment device 20, and a network 30.

The electrical treatment device 20 is a low-frequency treatment device that includes a plurality of electrodes to be brought into contact with a site of the body of a user, and performs treatment, such as easing shoulder stiffness of the user, by supplying a low-frequency pulse current to each electrode. The frequency of the low-frequency pulse current is, for example, from 1 Hz to 1200 Hz. However, the electrical treatment device 20 may be configured to use a pulse current of other frequency bands.

The electrical treatment device 20 is a cordless type device and includes a pad 2, a main body portion 4, and a holder that are integrated when used. The treatment is performed by combining these constituent elements. The main body portion functions as a control unit for controlling the electrical treatment device 20. The pad 2 has a sheet-like shape and is attached to a treatment site of the user (for example, a knee region).

The terminal device 10 is, for example, a smartphone provided with a touch panel 155, in which a touch sensor and a display are integrated. In the description hereinafter, a smartphone will be used as a representative example of the “terminal device”. However, the terminal device 10 may be another terminal device such as a tablet terminal device on which a touch panel is mounted.

The network 30 for connecting the terminal device 10 and the electrical treatment device 20 employs a short-range wireless communication system, typically Bluetooth (trade name) low energy (BLE). As such, the terminal device 10 and the electrical treatment device 20 are BLE devices having a function of performing wireless communication using BLE. However, the network 30 is not limited thereto, and a different wireless communication system, such as Bluetooth or a wireless local area network (LAN), may be employed.

A predetermined image is displayed on a display screen of the touch panel 155 of the terminal device 10. The predetermined image includes a plurality of electrode images. The plurality of electrode images respectively correspond to the plurality of electrodes formed in the pad 2 of the electrical treatment device 20. In the present embodiment, since the plurality of electrodes having a honeycomb shape are formed in the pad 2, each of the electrode images displayed on the touch panel 155 is arranged in a honeycomb-like pattern.

When an operation (touch operation) performed on the electrode image displayed on the touch panel 155 is received, the terminal device 10 instructs the electrical treatment device 20 to apply a pulse voltage to the electrode corresponding to the electrode image having been extracted in response to the touch operation. Specifically, the terminal device 10 includes a selection unit 403, an instruction unit 405, and a display control unit 407 as a main functional configuration.

In response to the touch operation, the display control unit 407 displays various operation screens for the electrical treatment device 20 on the display screen of the touch panel 155. The display control unit 407 arranges, on the touch panel 155, the electrode images respectively corresponding to the plurality of electrodes formed in the pad 2.

In response to the touch operations performed on the plurality of electrode images displayed on the touch panel 155, the selection unit 403 extracts a set of electrode images constituted of two electrode images among the plurality of electrode images, and selects a set of electrodes corresponding to the extracted set of electrode images.

The instruction unit 405 instructs the electrical treatment device 20 (for example, the main body portion 4) to apply a pulse voltage to the selected set of electrodes among the plurality of electrodes formed in the pad 2. Specifically, the instruction unit 405 transmits, via the network 30, instruction information indicating the above instruction to the electrical treatment device 20. When the polarity of one of the set of electrodes is positive, the polarity of the other electrode is negative. The electrical treatment device 20 performs treatment on the treatment site by applying a pulse voltage to the set of electrodes in accordance with the instruction information.

By touching the electrode images displayed on the touch panel 155 of the terminal device 10, the user can receive the treatment via the electrodes corresponding to the touched electrode images. In addition, the user can receive the treatment with a desired electrical stimulation pattern by continuously performing the touch operations on the touch panel 155 to sequentially select the electrode images. Since the electrode images corresponding to the electrodes formed in the pad 2 are displayed on the touch panel 155, the user can intuitively and easily select the electrode to be supplied with a pulse voltage.

### Configuration Example

### First Embodiment

### Hardware Configuration of Terminal Device 10

FIG. 2 is a block diagram illustrating an example of a hardware configuration of the terminal device 10 according to a first embodiment. The terminal device 10 includes, as main components, a processor 152, a memory 154, the touch panel 155, a wireless communication unit 160, a memory interface (I/F) 164, a communication interface (I/F) 166, a speaker 168, and a microphone 170.

The processor 152 typically may be an arithmetic processing unit such as a central processing unit (CPU) or a multi processing unit (MPU). The processor 152 functions as a control unit that controls the operation of components of the terminal device 10 by reading out and executing a program stored in the memory 154. By executing the program, the processor 152 realizes processing (steps) of the terminal device 10 described later.

The memory 154 is realized by a random access memory (RAM), a read-only memory (ROM), a flash memory, and the like. Programs executed by the processor 152, data used by the processor 152, and the like are stored in the memory 154.

The touch panel 155 is configured to be capable of detecting a touch position and a pressing force at the time of touch operation. The touch panel 155 is constituted of a display 156, a touch sensor 157 superimposed on the display 156, and a pressure sensor 158.

The display 156 is constituted of a liquid crystal display or the like, and configured to display various information in accordance with control from the processor 152. The touch sensor 157 detects a touch operation performed on the touch panel 155 by using a finger, a tablet pen or the like, along with a position (touch coordinates) in the touch panel 155, where the touch operation has been performed, and supplies the detected signal to the processor 152.

The pressure sensor 158 is, for example, a pressure sensor of a strain gauge scheme, and is provided at a portion of the display 156 to be distorted by the pressing force against the display screen. The processor 152 detects a pressing force against the display screen of the display 156 based on an output value from the pressure sensor 158. Note that the scheme for detecting the pressing force is not limited to a scheme using a pressure sensor, and may be another scheme as long as the scheme is able to detect the pressing force against an operation surface of the display 156.

The wireless communication unit 160 connects to a mobile communication network via a communication antenna 162 and transmits and receives signals for wireless communication. Accordingly, the terminal device 10 can communicate with other communication devices via a mobile communication network such as Long Term Evolution (LTE), for example.

The memory interface 164 reads data from an external storage medium 165. The processor 152 reads the data stored in the storage medium 165 via the memory interface 164 and stores the data in the memory 154. The processor 152 reads the data from the memory 154 and stores the data in the external storage medium 165 via the memory interface 164.

The storage medium 165 may also be media that store programs in a non-volatile manner, such as a compact disc (CD), digital versatile disk (DVD), Blu-ray (trade name) disc (BD), universal serial bus (USB) memory, and secure digital (SD) memory card.

The communication interface (I/F) 166 is a communication interface configured to exchange various data between the terminal device 10 and the electrical treatment device 20 and is realized by an adapter, a connector, or the like. As a communication scheme, for example, a wireless communication scheme such as Bluetooth low energy (BLE) or a wireless LAN may be employed.

The speaker 168 converts a sound signal from the processor 152 to sound and outputs the sound to the outside of the terminal device 10. The microphone 170 receives a sound input for the terminal device 10 and provides to the processor 152 a sound signal corresponding to the sound input.

### Configuration of Electrical Treatment Device 20

FIG. 3 is a perspective view illustrating an example of a configuration of the electrical treatment device 20 according to the first embodiment. FIG. 4 is a perspective view illustrating a state in which the main body portion 4 provided in the electrical treatment device 20 according to the first embodiment is separated from a holder 3 and the pad 2. FIG. 5 is a diagram illustrating an example of an electrode structure formed in the pad 2 according to the first embodiment.

Referring to FIGS. 3 and 4, the electrical treatment device 20 is a so-called cordless type low-frequency treatment device, and includes the pad 2, the holder 3, and the main body portion 4.

The pad 2 has a sheet-like shape and is configured to attach to the user's body. A conductive layer 2a is provided on a surface of a body-side portion 21 (lower surface), of the outer surfaces of the pad 2, that faces the body. The pad 2 is stuck to the user's skin by using a conductive gel or the like, and a low-frequency pulse current is supplied to the user through the conductive layer 2a.

Referring to FIG. 4, the pad 2 includes an attachment portion 2X and a treatment portion 2Y. The attachment portion 2X is held by the holder 3. A window portion 23 and a through hole 2H are provided at the attachment portion 2X. A positioning protrusion 312 of the holder 3 is disposed on the inside of the window portion 23. An interlock pin 33 of the holder 3 is inserted through the through hole 2H. The treatment portion 2Y is provided on both the left and right sides of the attachment portion 2X, and the conductive layer 2a is exposed on the body-side portion 21 of the treatment portion 2Y.

The conductive layer 2a is also exposed on the surface facing the main body portion 4 at the attachment portion 2X, and the exposed portion constitutes a pad side electrode portion 22. The pad side electrode portion 22 is formed to be electrically connected to a main body portion side electrode portion 43.

Referring to FIG. 5, the pad side electrode portion 22 includes electrode groups 61 and 65, in each of which a plurality of electrode portions are formed in a honeycomb-like pattern. The electrode group 61 is formed in one of the treatment portions 2Y, and the electrode group 65 is formed in the other one of the treatment portions 2Y. Each of the electrode groups 61 and 65 has nine electrodes, for example. A pulse voltage is applied between two electrodes in the electrode group 61 (or the electrode group 65). As a result, one electrode has a positive polarity and the other electrode has a negative polarity. Since the electrode groups 61 and 65 are each formed in the honeycomb-like pattern, the area of the treatment portion 2Y may be efficiently exploited. However, the electrode groups 61 and 65 are not limited to the honeycomb shape, and may be formed, for example, in a matrix pattern.

Referring to FIG. 4, the holder 3 includes a pad holding portion 31 with a plate-like shape and a pair of wall portions 32 erected from both ends of the pad holding portion 31. The attachment portion 2X of the pad 2 is disposed on an upper surface 311 of the pad holding portion 31. Double-sided adhesive tape, glue, adhesive, or the like is disposed, as necessary, between the upper surface 311 and the attachment portion 2X.

The positioning protrusion 312 is provided on the pad holding portion 31. By fitting the inner peripheral edge of the window portion 23 provided in the pad 2 to the positioning protrusion 312, the pad 2 can be positioned with respect to the holder 3. The interlock pin 33 is centrally disposed on the pad holding portion 31. When attaching the pad 2 to the holder 3, the interlock pin 33 is inserted into the through hole 2H.

The pad 2 is a consumable item, and the pad 2 can be detachably attached to the main body portion 4, thus allowing replacement of the pad 2. In the present embodiment, the holder 3 holds the pad 2 such that the holder 3 and the pad 2 are united, and the main body portion 4 is configured to be detachably attached to the pad 2 and the holder 3. The pad 2 can be replaced together with the holder 3, or it is also possible to reuse the holder 3 as necessary.

Referring to FIGS. 3 and 4, the main body portion 4 includes, as an outer cover, a case 4a formed in a substantially rectangular parallelepiped shape. A guiding/engagement portion 5 (FIG. 3) is formed between the case 4a and the holder 3, and the main body portion 4 (case 4a) is detachably attached to the holder 3. The guiding/engagement portion 5 is constituted by a protrusion 51 (FIG. 4) formed on a side surface 41 of the case 4a and a groove portion 52 (FIG. 4) formed in the wall portion 32 of the holder 3.

Referring to FIG. 4, the groove portion 52 includes a vertical groove portion 521 and a lateral groove portion 522. The vertical groove portion 521 is formed in the vertical direction and opens upward. The lateral groove portion 522 is formed in the lateral direction and opens at both ends. When the main body portion 4 is attached to the holder 3, both of the protrusion 51 and the groove portion 52 move closer together in a facing direction and engage together. By rotating and moving the main body portion 4 with respect to the holder 3, the engagement between the two is released, and the main body portion 4 can be removed from the holder 3.

The main body portion 4 includes a main body portion side electrode portion 43, a substrate (not illustrated), an electric circuit (not illustrated), and an interlock mechanism (not illustrated). The electric circuit includes various control devices and is mounted on the surface of the substrate. In a state in which the main body portion 4 is attached to the holder 3, an end portion of the main body portion side electrode portion 43 abuts the pad side electrode portion 22. Thus, the main body portion side electrode portion 43 and the pad side electrode portion 22 are electrically connected, whereby the electric circuit can supply a low-frequency pulse current to the pad side electrode portion 22.

A power source (not illustrated) such as a battery is also provided inside the main body portion 4 (case 4a). A switch 48S, a display portion (not illustrated) such as a light emitting diode (LED), and a button (not illustrated) are provided on the outside of the case 4a.

The control devices included in the main body portion 4 include a processor configured to execute various processing, a memory configured to store programs, data and the like, a communication interface configured to wirelessly communicate various types of data with the terminal device 10, and a waveform generation/output device configured to boost a power supply voltage and generate and output a low-frequency pulse current (treatment current).

The waveform generation/output device outputs a current that flows in a treatment site of the user's body via the pad 2. Specifically, when various operations for the electrical treatment device 20 are received via the touch panel 155, the terminal device 10 transmits a control signal corresponding to each of the operation contents to the electrical treatment device 20. The processor of the control devices of the electrical treatment device 20 receives the control signal from the terminal device 10 via the communication interface and instructs the waveform generation/output device to output a pulse voltage in accordance with the control signal. The waveform generation/output device outputs a pulse voltage waveform to the pad 2 according to the instruction.

FIG. 6 is a diagram for describing parameters of a pulse voltage waveform. Referring to FIG. 6, the parameters of the pulse voltage waveform include an amplitude (voltage) V, a pulse width W, and a pulse period T (that is, pulse frequency F=1/T). The waveform generation/output device can change treatment contents for the user by changing at least one parameter among the above-mentioned three parameters.

The waveform generation/output device typically boosts the power supply voltage to a predetermined voltage. The waveform generation/output device adjusts the boosted voltage to a voltage corresponding to a set amplitude. Specifically, the waveform generation/output device can adjust the amplitude of the pulse voltage at a predetermined number of levels (for example, 10 levels) in accordance with the instructions of the terminal device 10. For example, when a setting input of a predetermined level (for example, level 6) is received via the touch panel 155, the terminal device 10 instructs the waveform generation/output device to adjust the amplitude to a value corresponding to the predetermined level. The waveform generation/output device generates a treatment waveform (pulse waveform) corresponding to a treatment mode based on the adjusted amplitude, and outputs the generated treatment waveform to (the electrodes of) the pad 2.

The electrical treatment device 20 is provided with a plurality of treatment modes in advance. The treatment modes include, for example, “massage”, “tap”, “press”, and “sweep”. The waveform generation/output device can generate electrical stimulation corresponding to various modes including “massage”, “tap”, “press”, and “sweep” by changing pulse waveforms (including a pulse width, pulse interval, frequency, and output polarity). The waveform generation/output device adjusts electrical stimulation intensity by changing the amplitude of the pulse voltage. The waveform generation/output device may also adjust the electrical stimulation intensity by changing the pulse width.

### Operation Scheme

An operation scheme for the electrical treatment device 20 by using the terminal device 10 according to the first embodiment will be described. FIG. 7 is a diagram illustrating an example of an operation screen for selecting an electrode to be applied with a pulse voltage. Referring to FIG. 7, an operation screen 550 displayed on the touch panel 155 of the terminal device 10 includes an electrode group image 512 constituted of nine electrode images, and an electrode group image 514 constituted of nine electrode group images.

In the following description, regular hexagon-shaped electrode images to which the numbers from 1 to 18 are respectively assigned in FIG. 7 are each represented as an electrode image “number”. For example, in FIG. 7, the electrode image to which the numbered “1” is assigned is expressed as the electrode image “1”, and the electrode image to which the number “5” is assigned is expressed as the electrode image “5”. Accordingly, the electrode group image 512 is constituted of the electrode images “1” to “9”, and the electrode group image 514 is constituted of the electrode images “10” to “18”.

The electrode group image 512 is an image corresponding to the electrode group 61 (see FIG. 5) formed in one of the treatment portions 2Y of the pad 2 of the electrical treatment device 20, and the electrode group image 514 is an image corresponding to the electrode group 65 formed in the other one of the treatment portions 2Y. The electrode group images 512 and 514 are each arranged in a honeycomb-like pattern on the operation screen 550 in accordance with the actual electrode shape (that is, the honeycomb shape) of the electrode groups 61 and 65. Specifically, each electrode image in the electrode group image 512 is arranged in accordance with the arrangement scheme for each electrode of the electrode group 61, and each electrode image in the electrode group image 514 is arranged in accordance with the arrangement scheme for each electrode of the electrode group 65.

The operation screen 550 includes a honeycomb button 524 and a rectangle button 526. In the present embodiment, as illustrated in FIG. 5, the electrode groups 61 and 65 are honeycomb-shaped. Thus, on the operation screen 550, the electrode group images 512 and 514, in each of which each electrode is arranged in a honeycomb-like pattern, are displayed as a result of the honeycomb button 524 being selected. In a case where the electrode groups 61 and 65 are each formed in a rectangular shape (for example, a matrix pattern), the user selects the rectangle button 526, thereby making it possible to display the electrode group images 512 and 514, in each of which each electrode is arranged to form a rectangular shape, on the operation screen 550.

The operation screen 550 includes a replay button 516, a stop button 518, and a recording button 520. In the present embodiment, in a recording mode, the terminal device 10 is able to record a stimulation pattern corresponding to a series of touch operations received from a user (for example, touched electrode images, an order of touching the electrode images, and a pressing force of each touch), and replay (execute) the treatment corresponding to the recorded stimulation pattern. A pressing force display section 528 indicates a pressing force (for example, a pressing level “38”) when any electrode image is touched.

The replay button 516 is a button for replaying the recorded treatment contents, the stop button 518 is a button for stopping the replay or recording, and the recording button 520 is a button for causing the terminal device 10 to change over to the recording mode. Details of a replay scheme, stop scheme, and recording scheme will be described later.

Next, with reference to FIG. 7, a selection scheme for selecting two electrodes (that is, a set of electrodes) to be applied with a pulse voltage from the electrode groups 61 and 65 formed in the pad 2 will be described. In the electrical treatment device 20, a pulse voltage is applied between the selected two electrodes (that is, a positive polarity electrode and a negative polarity electrode) in the electrode groups. For ease of explanation, a scheme for selecting two electrodes in the electrode group 61 will be described, but the same applies to the electrode group 65.

With reference to FIG. 7, when the user touches a position Q in an internal region of the electrode image “8” of the electrode group image 512 corresponding to the electrode group 61, the electrodes corresponding to the electrode image “8” and the electrode image “9” respectively are automatically selected. Specifically, the terminal device 10 selects the electrodes respectively corresponding to the two electrode images closest to the touch coordinates of the position Q.

To be specific, the terminal device 10 calculates a distance between the touch coordinates of the position Q and the center coordinates of each electrode image (for example, the coordinates of the center point of the regular hexagon constituting the electrode image). The terminal device 10 extracts two electrode images in the ascending order of lengths of the calculated distances. In an example of FIG. 7, the electrode image “8” having the center coordinates closest in distance to the position Q and the electrode image “9” having the center coordinates second closest in distance to the position Q are extracted. The terminal device 10 instructs the electrical treatment device 20 to apply a pulse voltage between the electrode corresponding to the extracted electrode image “8” and the electrode corresponding to the extracted electrode image “9”.

As a result, only by touching the electrode images present at the desired positions in the electrode group image 512 without individually touching two electrode images, the user can receive electrical stimulation between the electrodes corresponding to the touched electrode images.

The terminal device 10 detects a pressing force (touch intensity) against the display screen of the touch panel 155 (display 156). The terminal device 10 can continuously detect the intensity of the pressing force when the display 156 is pressed by the touch operation. The terminal device 10 instructs the electrical treatment device 20 to apply a pulse voltage with a pulse width corresponding to the detected pressing force. The pressing force display section 528 indicates that the position Q is pressed by a pressing force corresponding to a pressing level “38”. Typically, as the pressing force is larger, the pulse width of the pulse voltage is set to be wider.

Thus, by strongly (or lightly) pressing an electrode image when a user touches the electrode image with his or her finger or the like, he or she can widen (or shorten) the pulse width of the pulse voltage to be applied to the electrode corresponding to the touched electrode image. In this way, the user can easily change the pulse width of the pulse voltage by adjusting the pressing force when performing the touch operation.

FIG. 8 is a diagram illustrating another example of an operation screen for selecting an electrode to be applied with a pulse voltage. FIG. 7 describes a configuration in which the electrodes respectively corresponding to two electrode images closest to the touch coordinates of the touch position are selected. FIG. 8 describes a configuration in which electrodes corresponding to electrode images closest to two touch positions, respectively, are selected.

With reference to FIG. 8, in an operation screen 560, an electrode image “8” closest to a touch position Q1 specified by a touch operation with a finger 610, and an electrode image “4” closest to a touch position Q2 specified by a touch operation with a finger 620 are extracted as a set of electrode images. The set of electrode images may be extracted by touching them with two fingers at the same time, or may be extracted by touching them with one finger in sequence. The terminal device 10 instructs the electrical treatment device 20 to apply a pulse voltage between the electrode corresponding to the extracted electrode image “4” and the electrode corresponding to the extracted electrode image “8”.

In an example of FIG. 8, the user can freely select two electrodes to be supplied with the pulse voltage by touching any two electrode images in the electrode group image 512. Thus, for example, the user can select two electrodes corresponding to two electrode images that are not adjacent to each other (for example, the electrode image “4” and electrode image “8”). When two electrodes that are not adjacent to each other are selected, electrical stimulation may be received across a wider range than in the case where two electrodes adjacent to each other are selected.

Next, an example in which electrodes are continuously selected by a series of touch operations will be described. FIG. 9 is a diagram illustrating an example of an operation screen in a case in which a series of touch operations is performed. Specifically, FIG. 9(a) is a diagram illustrating a first aspect of an operation screen 570. FIG. 9(b) is a diagram illustrating a second aspect after the first aspect illustrated in FIG. 9(a). FIG. 9(c) is a diagram illustrating a third aspect after the second aspect illustrated in FIG. 9(b). It is considered that a set of electrodes is selected in the selection scheme described in FIG. 7.

Referring to FIG. 9(a), in the operation screen 570, a user presses (touches) an internal region of an electrode image “7” with a finger 630. The terminal device 10 selects the electrodes respectively corresponding to the electrode image “7” and an electrode image “8”, and displays a pressing level “14” corresponding to a pressing force produced by the pressing with the finger 630 in a pressing force display section 528. The terminal device 10 instructs the electrical treatment device 20 to apply a pulse voltage with a pulse width corresponding to the pressing level “14” between the electrode corresponding to the electrode image “7” and the electrode corresponding to the electrode image “8”.

Subsequently, referring to FIG. 9(b), after the user presses the internal region of the electrode image “7” with the finger 630, the user performs a sliding operation in which the finger is moved to the vicinity of an electrode image “9”. In this case, the terminal device 10 selects the electrodes respectively corresponding to the electrode image “8” and the electrode image “9”, and displays a pressing level “20” corresponding to the pressing force in the pressing force display section 528.

The terminal device 10 instructs the electrical treatment device 20 to apply a pulse voltage with a pulse width corresponding to the pressing level “20” between the electrode corresponding to the electrode image “8” and the electrode corresponding to the electrode image “9”. Note that in FIG. 9(b), a locus 710 indicating the sliding operation by the user from the first aspect illustrated in FIG. 9(a) to the second aspect illustrated in FIG. 9(b).

Next, referring to FIG. 9(c), after having performed the sliding operation to the vicinity of the electrode image “9”, the user further performs a sliding operation to the vicinity of an electrode image “6”. In this case, the terminal device 10 selects the electrodes respectively corresponding to the electrode image “6” and the electrode image “9”, and displays a pressing level “32” corresponding to the pressing force in the pressing force display section 528.

The terminal device 10 instructs the electrical treatment device 20 to apply a pulse voltage with a pulse width corresponding to the pressing level “32” between the electrode corresponding to the electrode image “6” and the electrode corresponding to the electrode image “9”. Note that in FIG. 9(c), a locus 720 indicating the sliding operation by the user from the first aspect illustrated in FIG. 9(a) to the third aspect illustrated in FIG. 9(c).

From the above description, the user performs a series of touch operations (for example, sliding operations) for selecting sets of electrode images in the order of a set of electrode images including the electrode images “7” and “8”, a set of electrode images including the electrode images “8” and “9”, and a set of electrode images including the electrode images “9” and “6”. In addition, in the series of touch operations, the pressing force of the touching is gradually increased.

In response to the series of touch operations, the terminal device 10 sequentially executes the process for selecting the electrodes corresponding to the electrode images and the process for detecting the pressing forces, and sequentially transmits the instruction information based on the above selection process and detection process to the electrical treatment device 20. The electrical treatment device 20 applies a pulse voltage in accordance with the instruction information from the terminal device 10. With this, the instructions can be continuously given to the electrical treatment device 20 by the touch operations by using the terminal device 10, and as a result, it is possible to receive continuous electrical stimulation.

The user can grasp the selected electrodes in time series by checking the loci 710 and 720 of the touch operations, and can grasp a change in the electrical stimulation intensity (pulse width) in time series by checking the pressing levels displayed in the pressing force display section 528.

Next, a function of recording stimulation patterns will be described. Specifically, the terminal device 10 has a function of sequentially recording a set of electrodes (hereinafter, also referred to as “selected electrodes”) selected in response to a touch operation, the order of the selected electrodes, and the pressing force of the touch operation.

The terminal device 10 is considered to have received the selection of a recording button 520 from the user via the touch panel 155. In this case, the terminal device 10 transitions from a normal mode to a recording mode, and records a stimulation pattern corresponding to the touch operations. Assume that, for example, the user performs a series of touch operations as in the example of FIG. 9.

In response to the touch operation illustrated in FIG. 9(a), the terminal device 10 records the selected electrodes corresponding to the electrode images “7” and “8”, a selection order “1” of the selected electrodes, and the pressing level “14” indicating the pressing force at the time of selection. Subsequently, in response to the touch operation illustrated in FIG. 9(b), the terminal device 10 records the selected electrodes corresponding to the electrode images “8” and “9”, a selection order “2”, and the pressing level “20” indicating the pressing force. Then, in response to the touch operation illustrated in FIG. 9(c), the terminal device 10 records the selected electrodes corresponding to the electrode images “9” and “6”, a selection order “3”, and the pressing level “32” indicating the pressing force.

As described above, the terminal device 10 stores stimulation pattern information corresponding to the series of touch operations illustrated in FIGS. 9(a) to 9(c) in the memory 154. Typically, the stimulation pattern information includes information indicating the respective selected electrodes, the order of the respective selected electrodes, and pressing force information indicating the pressing forces at the time of selecting the respective selected electrodes.

The user selects a stop button 518 after the recording of the series of touch operations is ended. In response to receiving the input of the stop button 518, the terminal device 10 terminates the recording mode and transitions to the normal mode. Subsequently, when selection input of a replay button 516 is received, the terminal device 10 instructs the electrical treatment device 20 to execute treatment based on the recorded stimulation pattern information.

Specifically, the terminal device 10 instructs the electrical treatment device 20 to apply a pulse voltage with a pulse width corresponding to the pressing level “14” between the two electrodes corresponding to the electrode images “7” and “8” respectively. Subsequently, the terminal device 10 instructs the electrical treatment device 20 to apply a pulse voltage with a pulse width corresponding to the pressing level “20” between the two electrodes corresponding to the electrode images “8” and “9” respectively. Further, the terminal device 10 instructs the electrical treatment device 20 to apply a pulse voltage with a pulse width corresponding to the pressing level “32” between the two electrodes corresponding to the electrode images “9” and “6” respectively.

The terminal device 10 typically executes treatment based on stimulation pattern information at a speed corresponding to the actual series of touch operations in accordance with the stimulation pattern information, but the present invention is not limited thereto. For example, the terminal device 10 may execute the treatment at a slower speed than the speed corresponding to the actual series of touch operations (may perform a slow replay), or may perform the treatment at a faster speed than the above-mentioned speed (may perform a double-speed replay).

As described above, the user is allowed to record the desired stimulation pattern by performing a series of touch operations in the recording mode. In addition, the user can replay the desired stimulation pattern being recorded at any time by selecting the replay button 516, and therefore it is possible to omit the performing of the same touch operations again.

### Functional Configuration

FIG. 10 is a block diagram illustrating an example of a functional configuration of the terminal device 10 according to the first embodiment. Referring to FIG. 10, the terminal device 10 includes a touch position detection unit 401, the selection unit 403, the instruction unit 405, the display control unit 407, a pressing force detection unit 409, an adjustment unit 411, and an information storage unit 413.

The touch position detection unit 401 detects coordinate information when a touch operation is input, that is, coordinate information indicating a touch position on the touch panel 155 touched by the user in the touch operation. The touch position detection unit 401 acquires, in time series, changes in the coordinate information indicating the touch positions. Typically, the touch position detection unit 401 is implemented by the processor 152 and the touch sensor 157.

The selection unit 403 selects a set of electrodes constituted of two electrodes among a plurality of electrodes (for example, electrodes in the electrode group 61 or 65) in response to a touch operation performed on a predetermined image displayed on the touch panel 155 (for example, the electrode group image 512 or 514) based on the touch position detected by the touch position detection unit 401. Specifically, in response to the touch operation performed on the electrode group image 512 (or the electrode group image 514), the selection unit 403 extracts a set of electrode images constituted of two electrode images in the electrode group image, and selects a set of electrodes corresponding to the extracted set of electrode images. Typically, the selection unit 403 is implemented by the processor 152.

In a certain aspect, the selection unit 403 extracts, from the electrode group image 512 (or the electrode group image 514) displayed on the display screen of the touch panel 155, two electrode images closest to one touch position specified by the touch operation as a set of electrode images. Specifically, the selection unit 403 calculates distances from the coordinates of the one touch position to the center coordinates of each of the electrode images, identifies two electrode images in the ascending order of lengths of the calculated distances, and extracts the identified two electrode images as the set of electrode images.

In another aspect, the selection unit 403 extracts, from among the electrode images in the electrode group image 512 (or the electrode group image 514), an electrode image closest to a first touch position specified by a touch operation and an electrode image closest to a second touch position specified by the touch operation, as a set of electrode images.

The pressing force detection unit 409 detects a pressing force against the touch panel 155 by a touch operation. Typically, the pressing force detection unit 409 is implemented by the processor 152 and the pressure sensor 158.

The display control unit 407 arranges the electrode images indicating each of the electrode groups 61 and 65 on the display screen of the touch panel 155. Specifically, the display control unit 407 displays the operation screens 550 to 570 described above in accordance with the touch operations (and the touch positions). For example, the display control unit 407 displays, on the display 156, the loci 710 and 720, which reflects the touch operations. Typically, the display control unit 407 is implemented by the processor 152.

The adjustment unit 411 adjusts the electrical stimulation intensity provided to a site of the body of the user based on the pressing force when a set of electrodes is selected by the selection unit 403 in accordance with the touch operation. Specifically, the adjustment unit 411 increases the electrical stimulation intensity as the pressing force is larger.

For example, by adjusting the pulse width of a pulse voltage, the adjustment unit 411 adjusts the electrical stimulation intensity. In other words, the adjustment unit 411 increases the electrical stimulation intensity by widening the pulse width, and decreases the electrical stimulation intensity by shortening the pulse width. The adjustment unit 411 may adjust (for example, increase) the electrical stimulation intensity by adjusting (for example, increasing) the amplitude of the pulse voltage. Typically, the adjustment unit 411 is implemented by the processor 152.

The instruction unit 405 instructs the electrical treatment device 20 to apply a pulse voltage to a set of electrodes selected by the selection unit 403 from the electrode group 61 (or the electrode group 65). Typically, the instruction unit 405 is implemented by the processor 152.

In a certain aspect, the instruction unit 405 instructs the electrical treatment device 20 to apply a pulse voltage corresponding to the electrical stimulation intensity adjusted by the adjustment unit 411 to a set of electrodes.

In another aspect, the instruction unit 405 stores, in the information storage unit 413, stimulation pattern information in accordance with a series of touch operations in the recording mode in which the recording button 520 is selected. Typically, the stimulation pattern information includes each of sets of electrodes selected by the selection unit 403 in response to a series of touch operations, an order in which the sets of electrodes are selected, and pressing force information indicating pressing forces detected by the pressing force detection unit 409 when the sets of electrodes are selected. The pressing force information is information indicating a pulse width or a pressing force (for example, information corresponding to the “pressing level” in FIG. 7). The information storage unit 413 is implemented by the memory 154.

In response to the receiving of the selection of the replay button 516, the instruction unit 405 reads out the stimulation pattern information stored in the information storage unit 413, and instructs the electrical treatment device 20 to apply a pulse voltage to each set of electrodes based on the stimulation pattern information.

### Processing Procedure

FIG. 11 is a flowchart illustrating an example of a processing procedure of the terminal device 10 according to the first embodiment. Each step in FIG. 11 is mainly executed by the processor 152 of the terminal device 10. The processing in FIG. 11 is executed repeatedly at a predetermined period.

It is assumed that, at the start of the flowchart, an operation screen (for example, the operation screen 550) for giving a predetermined instruction to the electrical treatment device 20 is displayed on the display 156. For example, the terminal device 10 displays the operation screen by activating an operation application for operating the electrical treatment device 20.

Referring to FIG. 11, in response to a touch operation against the touch panel 155, the terminal device 10 extracts a set of electrode images from the electrode group images 512 and 514 displayed on the display screen, and selects a set of electrodes corresponding to the set of electrode images (step S12). For example, the terminal device 10 selects a set of electrodes in accordance with the selection scheme described in FIG. 7 or FIG. 8.

The terminal device 10 detects, via the touch panel 155, a pressing force against the display screen of the touch panel 155 when the set of electrodes is selected (step S14). The terminal device 10 adjusts a pulse width of a pulse voltage to be output from the electrical treatment device 20 based on the detected pressing force (step S16).

The terminal device 10 instructs the electrical treatment device 20 to apply a pulse voltage having the adjusted pulse width to the selected set of electrodes (step S18).

### Second Embodiment

In the first embodiment, the configuration is described in which the terminal device 10 and the electrical treatment device 20 are wirelessly connected and the electrical treatment device 20 performs treatment in accordance with instructions from the terminal device 10. In a second embodiment, a configuration will be described in which an electrical treatment device alone performs treatment on a user. In other words, in the second embodiment, the function of operating the electrical treatment device 20 by the terminal device 10 is enabled by the electrical treatment device itself.

### Configuration of Electrical Treatment Device

FIG. 12 is a diagram illustrating an example of an appearance of an electrical treatment device 200 according to the second embodiment. Referring to FIG. 12, the electrical treatment device 200 includes a control device 205, two pads 270 and 272, and a cord 280 for electrically connecting the control device 205 and the pads 270, 272.

The control device 205 is connected to the pads 270 and 272 by connecting a plug 282 of the cord 280 to plugs on the side of the pads 270 and 272 and inserting the cord 280 into a jack of the control device 205. The electrode group 61 illustrated in FIG. 5 is formed in the pad 270, and the electrode group 65 illustrated in FIG. 5 is formed in the pad 272.

A touch panel 230 is provided in the control device 205. The touch panel 230 is equivalent to the touch panel 155 described above. The control device 205 may be configured to further include, for example, other buttons, dials, and switches.

FIG. 13 is a block diagram illustrating an example of a hardware configuration of the electrical treatment device 200 according to the second embodiment. Referring to FIG. 13, the control device 205 of the electrical treatment device 200 includes a processor 210, a memory 220, the touch panel 230, a power source unit 240, and a waveform generation/output device 250. The control device 205 is connected to the pads 270 and 272.

The processor 210 is typically an arithmetic processing unit such as a CPU or an MPU. The processor 210 functions as a control unit that controls the operation of components of the electrical treatment device 200 by reading out and executing a program stored in the memory 220.

The memory 220 is achieved by a RAM, ROM, flash memory, or the like. The memory 220 stores programs executed by the processor 210, data used by the processor 210, and the like.

The touch panel 230 includes a display 231, a touch sensor 232, and a pressure sensor 233. The touch panel 230 has the same configuration and function as those of the touch panel 155 described in the first embodiment.

The power source unit 240 supplies power to each of components of the electrical treatment device 200. As the power source, an alkaline battery or a rechargeable battery, such as a lithium ion battery or nickel hydrogen battery may be used, for example. The power source stabilizes a battery voltage and generates a drive voltage that is supplied to each of the components.

The waveform generation/output device 250 has the same configuration and function as those of the waveform generation/output device included in the control device of the main body portion 4 described in the first embodiment.

### Functional Configuration

The electrical treatment device 200 has a functional configuration equivalent to a section including the touch position detection unit 401, the selection unit 403, the display control unit 407, the pressing force detection unit 409, the adjustment unit 411, and the information storage unit 413 in the functional configuration of the terminal device 10 illustrated in FIG. 10. Further, the electrical treatment device 200 includes, instead of the instruction unit 405 illustrated in FIG. 10, a treatment execution unit.

In the first embodiment, the user gives various instructions to the terminal device 10 via the touch panel 155, and then the instructions are transmitted from the terminal device 10 to the electrical treatment device 20, whereby the various instructions are indirectly given to the electrical treatment device 20. In the second embodiment, the user gives various instructions directly to the electrical treatment device 200 via the touch panel 230.

Specifically, the treatment execution unit of the electrical treatment device 200 performs treatment on a site of the body of the user by applying a pulse voltage to a set of electrodes selected by the selection unit 403 from the electrode group 61 (or the electrode group 65). In a certain aspect, the treatment execution unit applies a pulse voltage corresponding to the electrical stimulation intensity adjusted by the adjustment unit 411 to a set of electrodes.

In another aspect, the treatment execution unit stores, in the information storage unit 413, stimulation pattern information in accordance with a series of touch operations in the recording mode in which the recording button 520 is selected. In response to the receiving of the selection of the replay button 516, the treatment execution unit reads out the stimulation pattern information stored in the information storage unit 413, and applies a pulse voltage to each set of electrodes based on the stimulation pattern information.

### Other Embodiments

(1) In the embodiments described above, the configuration is described in which a plurality of electrode images (that is, the electrode group images 512 and 514) respectively corresponding to a plurality of electrodes (that is, the electrode groups 61 and 65) formed in the pad of the electrical treatment device are displayed, but the present invention is not limited to that configuration. For example, a configuration may be such that a single image including the overall shape of the electrode groups is displayed.

FIG. 14 is a diagram illustrating an example of an operation screen for selecting an electrode to be applied with a pulse voltage in another embodiment. Referring to FIG. 14, an operation screen 850 displayed on the touch panel 155 of the terminal device 10 contains an image 812 corresponding to the electrode group 61 (see FIG. 5) and an image 814 corresponding to the electrode group 65.

In the following description, regions formed in a regular hexagon shape and assigned with numbers 1 to 18 in FIG. 14 are each represented as a region “number”. For example, a region assigned with the number “1” in FIG. 14 is represented as a region “1”. As such, the image 812 includes regions “1” to “9” and the image 814 includes regions “10” to “18”. A solid line portion in the images 812 and 814 is displayed, but it is optional whether a dotted line portion and the numbers are displayed.

As a result of having selected a honeycomb button 524, the images 812 and 814 are depicted on the operation screen 850 in such a manner as to correspond to outer side shapes of the electrode groups 61 and 65 each having a honeycomb shape. In a case where the electrode groups 61 and 65 are each formed in a rectangular shape, the user selects a rectangle button 526, thereby causing the images 812 and 814 each having a rectangular shape to be displayed on the operation screen 850.

The plurality of regions “1” to “9” of the image 812 are respectively associated with the plurality of electrodes of the electrode group 61, and the plurality of regions “10” to “18” of the image 814 are respectively associated with the plurality of electrodes of the electrode group 65. Each region in the image 812 is arranged in accordance with the arrangement scheme for each electrode of the electrode group 61, and each region in the image 814 is arranged in accordance with the arrangement scheme for each electrode of the electrode group 65.

A selection scheme for the electrodes illustrated in FIG. 14 is basically the same as the selection scheme for the electrodes described in FIG. 7. To be specific, when the user touches a position Q5 of the region “8” in the image 812 corresponding to the electrode group 61 with a finger 830, the electrodes corresponding to the region “8” and the region “9” respectively are automatically selected. Specifically, the terminal device 10 calculates a distance from the touch coordinates of the position Q5 to the center coordinates of each of the regions, and extracts two regions “8” and “9” in the ascending order of lengths of the calculated distances. The terminal device 10 instructs the electrical treatment device 20 to apply a pulse voltage between the electrode corresponding to the region “8” and the electrode corresponding to the region “9”.

A selection scheme similar to the selection scheme for the electrodes as described in FIG. 8 may be employed. Specifically, the terminal device 10 extracts, in the image 812, a first region closest to a touch position specified by a touch operation with a certain finger and a second region closest to a touch position specified by a touch operation with another finger, as a set of regions. The terminal device 10 instructs the electrical treatment device 20 to apply a pulse voltage between the electrode corresponding to the first region and the electrode corresponding to the second region. Furthermore, the recording scheme and the replay scheme for the stimulation pattern in the case where the series of touch operations described in FIG. 9 is performed, may be similarly employed as well.

As discussed above, the selection unit 403 of the terminal device 10 according to the another embodiment extracts, in response to a touch operation onto a predetermined image (for example, the image 812 or 814), a set of regions constituted of two regions among the plurality of regions in the predetermined image, and then selects a set of electrodes corresponding to the set of regions.

In a certain aspect, the selection unit 403 extracts, from among the plurality of regions, two regions closest to a touch position specified by the touch operation as a set of regions. In another aspect, the selection unit 403 extracts, from among the plurality of regions, a first region closest to a first touch position specified by the touch operation and a second region closest to a second touch position specified by the touch operation, as a set of regions. Other functional configurations except for the selection unit 403 are the same as the functional configurations according to the first embodiment described above. The same configuration as the selection unit 403 according to the another embodiment may be employed in the electrical treatment device 200 according to the second embodiment.

(2) In the first embodiment discussed above, the configuration is described in which the terminal device 10 wirelessly communicates with the electrical treatment devices 20, but the present invention is not limited thereto. The terminal device 10 may be configured to communicate with the electrical treatment device 20 by wire using the universal serial bus (USB) or the like. In this case, the terminal device 10 and the main body portion of the electrical treatment device are so configured as to be capable of wire communication, and the main body portion and the pad may be connected with a cord or the like.

(3) In the embodiments described above, a program may be provided that causes a computer to function and execute controls such as those described in the flowchart described above. Such a program can also be provided as a program product stored on a non-temporary computer-readable recording medium attached to a computer, such as a flexible disk, a compact disc read only memory (CD-ROM), a secondary storage device, a main storage device, and a memory card. Alternatively, a program may be provided, which is stored on a recording medium such as a hard disk built into a computer. The program may also be provided by download via a network.

The configuration given as an example of the embodiment described above is an example configuration of the present invention. The configuration can be combined with other known technology, and parts thereof may be omitted or modified within the scope of the present invention. Furthermore, the processes and configurations of other embodiments may be employed as appropriate and implemented in the embodiments described above.

### Supplementary Notes

As described above, the present embodiment includes disclosures such as described below.

### Configuration 1

A terminal device (10) including a touch panel (155) and configured to be able to communicate with an electrical treatment device (20) including a plurality of electrodes to be brought into contact with a site of the body of a user, the terminal device including:

a display control unit (407) configured to display a predetermined image on a display screen of the touch panel;
a selection unit (403) configured to select a set of electrodes constituted of two electrodes among the plurality of electrodes in response to a touch operation performed on the predetermined image displayed on the display screen; and
an instruction unit (405) configured to instruct the electrical treatment device to apply a pulse voltage to the selected set of electrodes.

### Configuration 2

The terminal device (10) described in Configuration 1, wherein

the predetermined image includes a plurality of electrode images corresponding to the plurality of electrodes respectively, and
the selection unit (403) extracts a set of electrode images constituted of two electrode images among the plurality of electrode images in response to a touch operation performed on the plurality of electrode images, and selects the set of electrodes corresponding to the set of electrode images.

### Configuration 3

The terminal device (10) described in Configuration 2, wherein

the selection unit (403) extracts, from among the electrode images displayed on the display screen, two electrode images closest to a touch position specified by the touch operation as the set of electrode images.

### Configuration 4

The terminal device (10) described in Configuration 2, wherein

the selection unit (403) extracts, from among the electrode images, a first electrode image closest to a first touch position specified by the touch operation and a second electrode image closest to a second touch position specified by the touch operation, as the set of electrode images.

### Configuration 5

The terminal device (10) described in any one of Configurations 2 through 4, wherein

the plurality of electrodes are formed to have a honeycomb shape, and
each of the electrode images is arranged in a honeycomb-like pattern on the display screen of the touch panel.

### Configuration 6

The terminal device (10) described in Configuration 1, wherein

a plurality of regions in the predetermined image are associated with the plurality of electrodes respectively, and
the selection unit (403) extracts a set of regions constituted of two regions among the plurality of regions in response to a touch operation performed on the predetermined image, and selects the set of electrodes corresponding to the set of regions.

### Configuration 7

The terminal device (10) described in Configuration 6, wherein

the selection unit (403) extracts, from among the plurality of regions, two regions closest to a touch position specified by the touch operation as the set of regions.

### Configuration 8

The terminal device (10) described in Configuration 6, wherein

the selection unit (403) extracts, from among the plurality of regions, a first region closest to a first touch position specified by the touch operation and a second region closest to a second touch position specified by the touch operation, as the set of regions.

### Configuration 9

The terminal device (10) described in any one of Configurations 1 through 8, further including:

a pressing force detection unit (409) configured to detect a pressing force applied to the touch panel by the touch operation; and
an adjustment unit (411) configured to adjust an electrical stimulation intensity provided to the site based on the pressing force when the set of electrodes is selected, wherein
the instruction unit (405) instructs the electrical treatment device to apply a pulse voltage corresponding to the adjusted electrical stimulation intensity to the set of electrodes.

### Configuration 10

The terminal device (10) described in Configuration 9, wherein

the adjustment unit (411) increases the electrical stimulation intensity as the pressing force increases.

### Configuration 11

The terminal device (10) described in any one of Configurations 1 through 10, further including:

an information storage unit (413) configured to store pattern information including each of a plurality of the sets of electrodes selected by the selection unit, and an order in which the sets of electrodes are selected, in accordance with a series of the touch operations, wherein
the instruction unit (405) instructs the electrical treatment device to apply a pulse voltage to each of the sets of electrodes based on the pattern information stored in the information storage unit (413).

### Configuration 12

An electrical treatment device (200) having a touch panel (230), the electrical treatment device including:

a plurality of electrodes to be brought into contact with a site of the body of a user;
a display control unit (407) configured to display a predetermined image on a display screen of the touch panel (230);
a selection unit (403) configured to select a set of electrodes constituted of two electrodes among the plurality of electrodes in response to a touch operation performed on the predetermined image displayed on the display screen; and
a treatment execution unit configured to perform treatment on the site by applying a pulse voltage to the selected set of electrodes.

### Configuration 13

A treatment system (1000) including:

an electrical treatment device (20) including a plurality of electrodes to be brought into contact with a site of the body of a user; and
a terminal device (10) including a touch panel (155) and configured to be able to communicate with the electrical treatment device, wherein
the terminal device (10) further includes a display control unit (407) configured to display a predetermined image on a display screen of the touch panel (155), a selection unit (403) configured to select a set of electrodes constituted of two electrodes among the plurality of electrodes in response to a touch operation performed on the predetermined image displayed on the display screen, and an instruction unit (405) configured to instruct the electrical treatment device to apply a pulse voltage to the selected set of electrodes.

The embodiments described herein are illustrative in all respects and are not intended as limitations. The scope of the present invention is indicated not by the descriptions above but by the claims and includes all meaning equivalent to the scope and changes within the scope.

### REFERENCE SIGNS LIST

2, 270, 272 Pad
2H Through hole
2X Attachment portion
2Y Treatment portion
2a Conductive layer
3 Holder
4 Main body portion
4a Case
5 Guiding/engagement portion
10 Terminal device
20, 200 Electrical treatment device
21 Body-side portion
22 Pad side electrode portion
23 Window portion
30 Network
31 Pad holding portion
32 Wall portion
33 Interlock pin
41 Side surface
43 Main body portion side electrode portion
48S Switch
51 Protrusion
52 Groove portion
61, 65 Electrode group
152, 210 Processor
154, 220 Memory
155, 230 Touch panel
156, 231 Display
157, 232 Touch sensor
158, 233 Pressure sensor
160 Wireless communication unit
162 Communication antenna
164 Memory interface
165 Storage medium
168 Speaker
170 Microphone
205 Control device
240 Power source unit
250 Waveform generation/output device
280 Cord
282 Plug
311 Upper surface
312 Positioning protrusion
401 Touch position detection unit
403 Selection unit
405 Instruction unit
407 Display control unit
409 Pressing force detection unit
411 Adjustment unit
413 Information storage unit
512, 514 Electrode group image
516 Replay button
518 Stop button
520 Recording button
521 Vertical groove portion
522 Lateral groove portion
524 Honeycomb button
526 Rectangle button
528 Pressing force display section
530, 550, 560, 570 Operation screen
610, 620, 630, 830 Finger
710, 720 Locus
812, 814 Image
1000 Treatment system

1. A terminal device including a touch panel and configured to be able to communicate with an electrical treatment device including a plurality of electrodes to be brought into contact with a site of a body of a user, the terminal device comprising:
a display control unit configured to display a predetermined image on a display screen of the touch panel;
a selection unit configured to select a set of electrodes constituted of two electrodes among the plurality of electrodes in response to a touch operation performed on the predetermined image displayed on the display screen; and
an instruction unit configured to instruct the electrical treatment device to apply a pulse voltage to the selected set of electrodes, wherein
a plurality of regions in the predetermined image are associated with the plurality of electrodes respectively, and
the selection unit extracts a set of regions constituted of two regions among the plurality of regions in response to a touch operation performed on the predetermined image, selects the set of electrodes corresponding to the set of regions, and extracts, from among the plurality of regions, two regions closest to a touch position specified by the touch operation as the set of regions.

2. The terminal device according to claim 1, further comprising:
a pressing force detection unit configured to detect a pressing force applied to the touch panel by the touch operation; and
an adjustment unit configured to adjust an electrical stimulation intensity provided to the site based on the pressing force when the set of electrodes is selected, wherein
the instruction unit instructs the electrical treatment device to apply a pulse voltage corresponding to the adjusted electrical stimulation intensity to the set of electrodes.

3. The terminal device according to claim 2, wherein
the adjustment unit increases the electrical stimulation intensity as the pressing force increases.

4. The terminal device according to claim 1, further comprising:
an information storage unit configured to store pattern information including each of a plurality of the sets of electrodes selected by the selection unit, and an order in which the sets of electrodes are selected, in accordance with a series of the touch operations, wherein
the instruction unit instructs the electrical treatment device to apply a pulse voltage to each of the sets of electrodes based on the pattern information stored in the information storage unit.

5. An electrical treatment device including a touch panel, the electrical treatment device comprising:
a plurality of electrodes to be brought into contact with a site of a body of a user;
a display control unit configured to display a predetermined image on a display screen of the touch panel;
a selection unit configured to select a set of electrodes constituted of two electrodes among the plurality of electrodes in response to a touch operation performed on the predetermined image displayed on the display screen; and
a treatment execution unit configured to perform treatment on the site by applying a pulse voltage to the selected set of electrodes, wherein
a plurality of regions in the predetermined image are associated with the plurality of electrodes respectively, and
the selection unit extracts a set of regions constituted of two regions among the plurality of regions in response to a touch operation performed on the predetermined image, selects the set of electrodes corresponding to the set of regions, and extracts, from among the plurality of regions, two regions closest to a touch position specified by the touch operation as the set of regions.

6. A treatment system comprising:
an electrical treatment device including a plurality of electrodes to be brought into contact with a site of a body of a user; and
a terminal device including a touch panel and configured to be able to communicate with the electrical treatment device, wherein
the terminal device further includes,
a display control unit configured to display a predetermined image on a display screen of the touch panel,
a selection unit configured to select a set of electrodes constituted of two electrodes among the plurality of electrodes in response to a touch operation performed on the predetermined image displayed on the display screen, and
an instruction unit configured to instruct the electrical treatment device to apply a pulse voltage to the selected set of electrodes,
a plurality of regions in the predetermined image are associated with the plurality of electrodes respectively, and
the selection unit extracts a set of regions constituted of two regions among the plurality of regions in response to a touch operation performed on the predetermined image, selects the set of electrodes corresponding to the set of regions, and extracts, from among the plurality of regions, two regions closest to a touch position specified by the touch operation as the set of regions.

7. A terminal device including a touch panel and configured to be able to communicate with an electrical treatment device including a plurality of electrodes to be brought into contact with a site of a body of a user, the terminal device comprising:
a display control unit configured to display a predetermined image on a display screen of the touch panel;
a selection unit configured to select a set of electrodes constituted of two electrodes among the plurality of electrodes in response to a touch operation performed on the predetermined image displayed on the display screen;
an instruction unit configured to instruct the electrical treatment device to apply a pulse voltage to the selected set of electrodes; and
an information storage unit configured to store pattern information including each of a plurality of the sets of electrodes selected by the selection unit, and an order in which the sets of electrodes are selected, in accordance with a sliding operation which is a series of the touch operations, wherein
the instruction unit instructs the electrical treatment device to apply a pulse voltage to each of the sets of electrodes based on the pattern information stored in the information storage unit, and
the display control unit displays a locus indicating the sliding operation on the display screen.

8. The terminal device according to claim 2, further comprising:
an information storage unit configured to store pattern information including each of a plurality of the sets of electrodes selected by the selection unit, and an order in which the sets of electrodes are selected, in accordance with a series of the touch operations, wherein
the instruction unit instructs the electrical treatment device to apply a pulse voltage to each of the sets of electrodes based on the pattern information stored in the information storage unit.

9. The terminal device according to claim 3, further comprising:
an information storage unit configured to store pattern information including each of a plurality of the sets of electrodes selected by the selection unit, and an order in which the sets of electrodes are selected, in accordance with a series of the touch operations, wherein
the instruction unit instructs the electrical treatment device to apply a pulse voltage to each of the sets of electrodes based on the pattern information stored in the information storage unit.
